SEQUENCE LISTING (4 sequences)

SEQ ID NO: 1 - protein, 6 aa, Artificial Sequence
  Description of Artificial Sequence Synthetic peptide
HWKWFK

SEQ ID NO: 2 - protein, 6 aa, Artificial Sequence
  Description of Artificial Sequence Synthetic peptide
HWRWFK

SEQ ID NO: 3 - protein, 6 aa, Artificial Sequence
  Description of Artificial Sequence Synthetic peptide
HWHWFK

SEQ ID NO: 4 - protein, 6 aa, Artificial Sequence
  Description of Artificial Sequence Synthetic peptide
HWAWFK

(19) United States
(12) Patent Application Publication
(10) Pub. No.: US 2011/0143992 A1
(43) Pub. Date: Jun. 16, 2011

(54) Methods and Compositions Related to GHS-R Antagonists

(76) Inventors: Dennis Taub, Baltimore, MD (US); Vishwa Deep Dixit, Baton Rouge, LA (US)

(21) Appl. No.: 12/223,908

(22) PCT Filed: Feb. 13, 2007
(86) PCT No.: PCT/US2007/004101

§ 371 Date: Feb. 3, 2011

Related U.S. Application Data
(60) Provisional application No. 60/773,076, filed on Feb. 13, 2006.

(51) Int. Cl. A61K 38/08; A61P 5/00; A61P 11/06; A61P 9/10; A61P 31/04; A61P 31/12; A61P 31/18; A61P 31/22; A61P 1/16; A61P 7/12; A61P 19/02; A61P 25/28; A61P 17/00; A61P 35/00

(52) U.S. Cl. 514/1.4; 514/1.7; 514/1.9; 514/2.4; 514/2.6; 514/2.7; 514/2.8; 514/3.7; 514/3.8; 514/4.2; 514/4.3; 514/6.9; 514/16.6; 514/17.8; 514/18.7; 514/19.3; 514/19.4; 514/19.5; 514/19.8; 514/21.8

(57) ABSTRACT

Disclosed herein are methods and compositions related to GHS-R antagonists.

### CROSS-REFERENCE TO RELATED APPLICATIONS

This application claims the benefit of U.S. provisional application No. 60/773,076 filed on Feb. 13, 2006. The aforementioned application is herein incorporated by this reference in its entirety.

### SUMMARY OF THE INVENTION

The present invention provides methods of blocking binding to chemokine receptors.

Also provided by the present invention are methods of treating or preventing a viral infection, comprising administering an effective amount of a GHS-R antagonist.

Also provided by the present invention are methods of treating or preventing cancer, comprising administering an effective amount of a GHS-R antagonist.

Also provided by the present invention are methods of treating or preventing inflammation, comprising administering an effective amount of a GHS-R antagonist.

Also provided by the present invention are methods of treating or preventing atherosclerosis, comprising administering an effective amount of a GHS-R antagonist such as D-Lys3-GHRP-6.

Also provided by the present invention are methods of blocking HIV entry and infectivity of chemokine receptor-expressing cells using an effective amount of a GHS-R antagonist such as D-Lys3-GHRP-6.

Also provided by the present invention are methods of stem cell mobilization for transplantation in subjects with multiple myeloma and non-Hodgkin's lymphoma, comprising administering an effective amount of a GHS-R antagonist such as D-Lys3-GHRP-6.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows the chemical structure of D-Lys3-GHRP-6 (DLG).

FIG. 2 shows that D-Lys3-GHRP-6 inhibits SDF-1 induced intracellular calcium release from human T cells. Unstimulated primary human T cells were loaded with Fura-2AM and treated with SDF-1, DLG did not flux calcium by itself but led to a dose dependent inhibition of SDF-1 induced calcium release.

FIG. 3 shows DLG inhibits CCR5 mediated calcium release. Fura2 AM labeled CEM-R5 cells were treated with MIP-1β (100 ng/ml) along with DLG (10⁻² M) and data is presented as percent inhibition of calcium release by DLG post MIP-1β treatment.

FIG. 4 shows DLG does not affect CCR7 and EDG receptor signaling. MIP-3β induced calcium release (A) and sphingosine 1 phosphate (B) is not affected by DLG treatment in human T cells.

FIG. 5 shows DLG inhibits SDF-1 and MIP-1β binding. T-SUP1 lymphoma cells were treated with DLG (10⁻² M) for 15 minutes and then utilized for ligand binding assay using FITC labeled (A) SDF-1 and (13) MIP-3β and mean fluorescence intensity was plotted as (C) percent maximal binding.

FIG. 6 shows DLG inhibits SDF-1 induced chemotaxis. Primary human T cells and CEM-R5 cells labeled with Hoechst33342 were treated with SDF-1 and DLG and placed in Transwell chambers. DLG dose dependently inhibited the SDF-1 induced migration in human T (A) and CEMR5 (B) cells.

FIG. 7 shows DLG inhibits SDF-1 mediated signaling in human astrocytoma cells. SDF-1 treatment in SW1008 and U118 cells induces ERK phosphorylation within 5 minutes, DLG pretreatment for 30 min abrogates SDF-1 induced ERK activation.

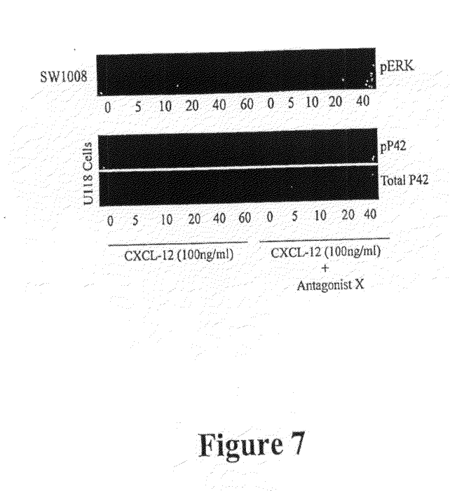

FIG. 8 shows DLG inhibits HIV-1 infectivity of CD4+ CXCR4+ human CEM T cell line. DLG pretreatment (30 minutes at 1 ug/ml) in CEM-GFP cells inhibits HIV-1-induced GFP expression. CEM-GFP can be used to monitor infection with HIV-1 (CXCR4, SI strains), and HIV-2. Productive infection generates green fluorescent protein (GFP). A control containing SDF-1 alone also demonstrated similar levels of inhibition of HIV infectivity.

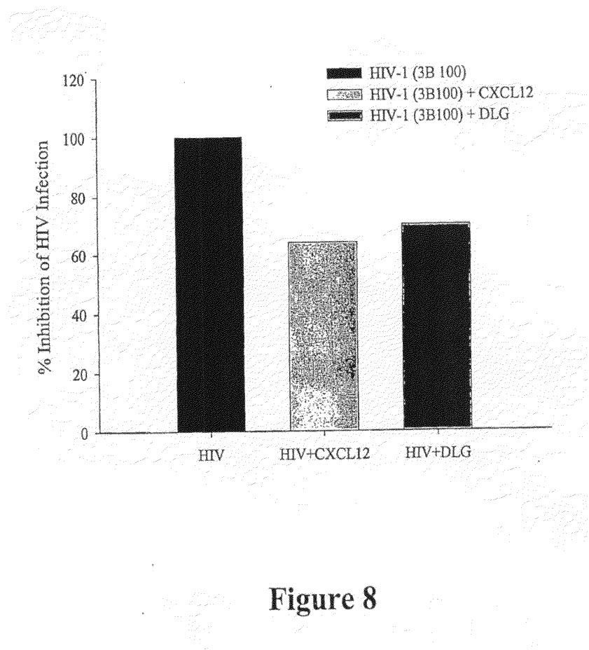

### DETAILED DESCRIPTION OF THE INVENTION

The present invention may be understood more readily by reference to the following detailed description of preferred embodiments of the invention and the Examples included therein and to the Figures and their previous and following description.

### A. Definitions

Before the present methods and compositions are disclosed and described, it is to be understood that this invention is not limited to specific methods or specific substances unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a substance” includes one or more substances, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

The terms “higher,” “increases,” “elevates,” or “elevation” refer to increases above basal levels, or as compared to a control. The terms “low,” “lower,” “inhibits,” “inhibition,” “reduces,” or “reduction” refer to decreases below basal levels, or as compared to a control. For example, basal levels are normal in vivo levels prior to, or in the absence of, inflammation or the addition of an agent which causes inflammation.

The terms “mediate” or “mediation” and “modulate” or “modulation” mean to regulate, or control, in particular to increase, enhance, elevate, or alternatively to lower, inhibit, or reduce. The terms “mediate” and “modulate” are used interchangeably throughout.

“Inflammation” or “inflammatory” is defined as the reaction of living tissues to injury, infection, or irritation. Anything that stimulates an inflammatory response is said to be inflammatory.

“Inflammatory disease” is defined as any disease state associated with inflammation.

“Infection” or “infectious process” is defined as one organism being invaded by any type of foreign material or another organism. The results of an infection can include growth of the foreign organism, the production of toxins, and damage to the host organism. Infection includes viral, bacterial, parasitic, and fungal infections, for example.

“Cancer therapy” is defined as any treatment or therapy useful in preventing, treating, or ameliorating the symptoms associated with cancer. Cancer therapy can include, but is not limited to, apoptosis induction, radiation therapy, and chemotherapy.

As used throughout, by a “subject” is meant an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

The terms “control levels” or “control cells” are defined as the standard by which a change is measured, for example, the controls are not subjected to the experiment, but are instead subjected to a defined set of parameters, or the controls are based on pre- or post-treatment levels.

By “treating” is meant that an improvement in the disease state, i.e., the inflammatory response, is observed and/or detected upon administration of a substance of the present invention to a subject. Treatment can range from a positive change in a symptom or symptoms of the disease to complete amelioration of the inflammatory response (e.g., reduction in severity or intensity of disease, alteration of clinical parameters indicative of the subject's condition, relief of discomfort or increased or enhanced function), as detected by art-known techniques.

By “preventing” is meant that after administration of a substance of the present invention to a subject, the subject does not develop the symptoms of a disclosed condition.

### B. General

Chemokines are small peptides that are known to exert potent regulatory effects on migration and activation of various immune and non hematopoietic cells via ligation to their seven transmembrane G-protein coupled receptors (Miyasaka et al. Nat Rev Immunol. 5:360-370 (2004), Balkwill F. Nat Rev Cancer 7:540-550 (2004)). The CXC chemokine SDF-1 or CXCL-12 is highly expressed in bone marrow stromal cells and potently stimulates the migration of T cells and monocytes via interactions with CXCR4 receptor (Campbell et al. Immunol Rev. 195:58-71 (2003)). The CXCR4 receptor is widely expressed on hematopoietic stem cells, monocytes, T and B lymphocytes (Cascieri and Springer Curr Opin Chem Biol. 4:420-427 (2000)). CCR5 another GPC chemokine receptor serves as an endogenous ligand for CC chemokines, MIP-1α/CCL3, MIP-1β/CCL4 and RANTES/CCL5. CC chemokine receptor 5 (CCR5) regulates trafficking and effector functions of memory/effector T-lymphocytes, macrophages, and immature dendritic cells (Cascieri and Springer (2000)). Interestingly, chemokine receptors CXCR4 and CCR5 have attracted substantial interest because they form portals of cellular entry for the human immunodeficiency viruses (HIV-1 and HIV-2) and related simian or feline retroviruses (Castagna et al. Drugs. 65: 879-904 (2005)). While all the HIV-1 strains require CD4 to enter and infect cells, some use the chemokine receptor CXCR4 (T-tropic/X4 strain or syncytium-inducing viruses), or CCR5 (M-tropic/R5 strain or non-syncytium-inducing viruses) and some can utilize either coreceptor (R5X4 strains) for these purposes. In addition to pathogenesis of HIV, CXCR4 and CCR5 receptors have been implicated in motility, invasion and metastasis of a wide variety of cancer cell types (Balkwill F. (2004)). Given the involvement of CXCR4 and CCR5 in HIV, cancers and inflammation these receptors have emerged as potential targets for therapeutic intervention (Castagna et al. (2005)).

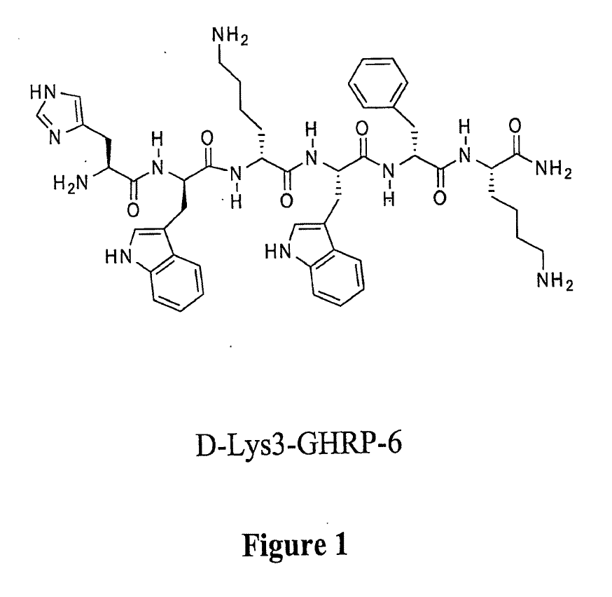

Growth hormone secretagogue receptor (GHS-R) belongs to a seven transmembrane GPCR family and serves as an endogenous ligand for stomach derived hormone ghrelin (Howard et al. Science 273, 974-977 (1996), Kojima et al. Physiol. Rev. 85, 495-522 (2005)). Growth hormone releasing peptide-6 (GHRP-6) is one of earliest synthetic peptidyl GHS-R agonists utilized to study the functions of GHS-R prior to the discovery of the endogenous ligand ghrelin (Smith R G. Endocr Rev. 26: 346-360 (2005)). Modification of GHRP-6 (H-His-D-Trp-Ala-Trp-D-Phe-Lys-NH2) from alanine to D-lysine resulted in a GHS-R. antagonist D-[Lys3]GHRP-6 (H-His-D-Trp-D-Lys-Trp-D-Phe-Lys-NH2). Currently, the D-[Lys3]GHRP-6 (DLG) is utilized in in vitro and in vivo studies as a selective GHS-R antagonist (Kojima et al. (2005)) (FIG. 1). However, no studies have yet addressed the specificity and efficacy of this compound in human T lymphocytes or T cell lines. Given the potent effects of natural GHS-R ligand ghrelin on human T cell responses (Rubin et al. Proc Natl Acad Sci USA. 100:13513-13518 (2003)), the specificity of DLG and its potential interactions with other immunologically relevant GPCRs of chemokine family were investigated, and it is shown herein that DLG antagonizes CXCR4 and CCR5 receptors.

Chemokines and their receptors also play an important role in immune and inflammatory responses by regulating the directional migration and activation of leukocytes. These molecules have also been implicated in hematopoiesis, angiogenesis, embryonic development, and breast cancer metastasis. In addition, chemokine receptors such as CXCR4 and CCR5 have been shown to act as co-receptors for the entry and infection of HIV-1 and HIV-2.

CC chemokines MIP-1α (CCL3), MIP-1β(CCL4), and RANTES¹(CCL5), the cognate ligands for CCR5, have been shown to inhibit HIV infection in vitro. Subsequently, stromal-derived factor-1α (SDF-1α/CXCL12), the cognate ligand for the CXCR4 receptor, was also shown to inhibit infection by T-tropic viruses. It has been demonstrated that the ligand-induced endocytosis of CCR5 and CXCR4 plays an important role in inhibiting HIV entry into the cells. Furthermore, effective anti-HIV activity of the chemically modified form of the CC chemokines correlates with the ability of these ligands to induce irreversible and efficient down-regulation of CCR5.

Receptor phosphorylation-dependent and -independent mechanisms have been shown to regulate CXCR4 receptor internalization. The cytoplasmic tail of CCR5 has been shown to play a major role in receptor internalization and signaling. A degradation motif was identified in the C-terminal domain of CXCR4. Moreover, the agonist-mediated ubiquitination of the CXCR4 receptor was observed to be blocked when the lysine residues in this degradation motif were mutated. It has also been observed that the proteasome pathway plays a major role in the down-modulation of these receptors (Femandis et al., 2002).

CXCR4 and CCR5 have also been implicated in crucial processes such as ovulation, menstruation, embryo implantation, parturition and pathological processes such as preterm delivery, HIV infection, endometriosis and ovarian hyperstimulation syndrome (Dominguez et al., 2003; Cocchi et al., 1995; Simón et al., 1998). A specific molecular crosstalk between embryo and endometrium has been reported during the human implantation process (Glasser et al., 1991; De los Santos et al., 1996). The endometrial epithelium is an important element where the molecular interactions between the embryo and the endometrium seem to be initiated (Simón et al., 1997; Galan et al., 2000; Meseguer et al., 2001). The endometrial epithelium produces and secretes chemokines (Arici et al., 1998; Caballero-Campo et al., 2002).

### D. Compositions

Disclosed are the components to be used to prepare the disclosed compositions as well as the compositions themselves. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular peptide is disclosed and discussed and a number of modifications that can be made to a number of molecules including the peptide are discussed, specifically contemplated is each and every combination and permutation of the amino acids within the peptide and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-B, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. GHRP-6

Growth hormone (GH) secretion is regulated by two hypothalamic peptides: GH-releasing hormone (GHRH), which exerts stimulatory effect on GH release and somatostatin which exhibits an inhibitory influence. It has been demonstrated that GH secretion can also be stimulated by synthetic oligopeptides termed GH-releasing peptides (GHRP) such as hexarelin and various hexarelin analogs (Ghigo et al., European Journal of Endocrinology, 136, 445-460, 1997). These compounds act through a mechanism which is distinct from that of GHRH (C. Y. Bowers, in “Xenobiotic Growth Hormone Secretagogues”, Eds. B. Bercu and R. F. Walker, Pg. 9-28, Springer-Verlag, N.Y. 1996) and by interaction with specific receptors localized in the hypothalamus and pituitary gland ((a) G. Muccioli et al., Journal of Endocrinology, 157, 99-106, 1998; (b) G. Muccioli, “Tissue Distribution of GHRP Receptors in Humans”, Abstracts IV European Congress of Endocrinology, Sevilla, Spain, 1998). It has also been demonstrated that GHRP receptors are present not only in the hypothalamo-pituitary system but even in various human tissues not generally associated with GH release.

GHRPs and their antagonists are described, for example, in the following publications: C. Y. Bowers, supra, R. Deghenghi, “Growth Hormone Releasing Peptides”, ibidem, 1996, pg. 85-102; R. Deghenghi et al., “Small Peptides as Potent Releasers of Growth Hormone”, J. Ped. End. Metab., 8, pg. 311-313, 1996; R. Deghenghi, “The Development of Impervious Peptides as Growth Hormone Secretagogues”, Acta Paediatr. Suppl., 423, pg. 85-87, 1997; K. Veeraraganavan et al., “Growth Hormone Releasing Peptides (GHRP) Binding to Porcine Anterior Pituitary and Hypothalamic Membranes”, Life Sci., 50, Pg. 1149-1155, 1992; and T. C. Somers et al., “Low Molecular Weight Peptidomimetic Growth Hormone Secretagogues, WO 96/15148 (May 23, 1996). The GHRPs and growth hormone secretagogues are considered as a second generation product destined to replace in the near future the uses of GH in most instances. Accordingly, the use of GHRPs and growth hormone secretagogues present a number of advantages over the use of GH per se.

GH-releasing peptide GHRP-6 is a synthetic compound that releases GH in a specific and dose-related manner that is different from those of growth hormone releasing hormone (GHRH). In humans, GHRP-6 is more efficacious than GHRH, and a striking synergistic action on GH release is observed when GHRP-6 and GHRH administered simultaneously. Based such a synergistic action, it has been hypothesized that GHRP-6 acts through a double mechanism by actions exerted both at the pituitary and hypothalamic levels.

The term “GHS-R antagonist” is used throughout to refer to any molecule (or functional fragment thereof) that functions as an antagonist of GHS-R. For example, there are multiple variations of GHRP-6 that can be used with the methods disclosed herein as an antagonist. Examples include, but are not limited to, and D-[Lys3]GHRP-6 (H-His-D-Trp-D-Lys-Trp-D-Phe-Lys-NH2, SEQ ID NO: 1); D-[Arg3]GHRP-6 (H-His-D-Trp-D-Arg-Trp-D-Phe-Lys-NH2, SEQ ID NO: 2); D-[His3JGHRP-6 (H-His-D-Trp-D-His-Trp-D-Phe-Lys-NH2, SEQ ID NO: 3); and D-[Ala3]GHRP-6(H-His-D-Trp-D-Ala-Trp-D-Phe-Lys-NH2, SEQ ID NO: 4).

2. Homology/Identity

It is understood that one way to define any known variants and derivatives or those that might arise, of the disclosed genes and proteins herein is through defining the variants and derivatives in terms of homology to specific known sequences. The term “GHRP” is used throughout to refer to any GH-releasing peptide molecule or functional fragment thereof. “Fragment” is defined as any subpart of the reference sequence. The methods of the invention include using full length GHS-R antagonist, such as SEQ ID NO: 1, for example, as well as functional fragments thereof. Also included are sequences longer than SEQ ID NO: 1 and include amino acids before and/or after the functional GHRP-6 molecule.

Also specifically disclosed are variants of these and other proteins herein disclosed which have at least, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, (1989), Jaeger et al. Proc. Natl. Acad. Sci. USA 86:7706-7710 (1989), Jaeger et al. Methods Enzymol. 183:281-306 (1989) which are herein incorporated by reference for at least material related to nucleic acid alignment.

3. Nucleic Acids

There are a variety of molecules disclosed herein that are nucleic acid based, including for example the nucleic acids that encode, for example, GHRP-6 as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a. Nucleotides and Related Molecules

A nucleotide is a molecule that contains a base moiety, a sugar moiety and a phosphate moiety. Nucleotides can be linked together through their phosphate moieties and sugar moieties creating an intemucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to any of the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 86, 6553-6556 (1989)).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b. Sequences

There are a variety of sequences related to, for example, GHRP-6, as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

4. Peptides

a. Peptide Variants

As discussed herein there are numerous variants of GHS-R antagonists that are known and herein contemplated. In addition, to the known functional GHRP-6 species variants there are derivatives of GHRP-6 which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| alanine | Ala | A |
| arginine | Arg | R |
| asparagine | Asn | N |
| aspartic acid | Asp | D |
| cysteine | Cys | C |
| glutamic acid | Glu | E |
| glutamine | Gln | K |
| glycine | Gly | G |
| histidine | His | H |
| isolelucine | Ile | I |
| leucine | Leu | L |
| lysine | Lys | K |
| phenylalanine | Phe | F |
| proline | Pro | P |
| serine | Ser | S |
| threonine | Thr | T |
| tyrosine | Tyr | Y |
| tryptophan | Trp | W |
| valine | Val | V |

TABLE 2
Amino Acid Substitutions
Original Residue Exemplary Conservative
Substitutions, others are known in the art

Ala and Ser
Arg and Lys, Gln
Asn and Gln, His
Asp and Glu
Cys and Ser
Gln and Asn, Lys
Glu and Asp
Gly and Pro
His and Asn, Gln
Ile and Leu, Val
Leu and Ile, Val
Lys and Arg; Gln
Met and Leu, ile
Phe and Met, Leu, Tyr
Ser and Thr
Thr and Ser
Trp and Tyr
Tyr and Trp, Phe
Val and Ile, Leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 (1983)), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NOS: 1-4 set forth particular sequences of GHRP-6. Specifically disclosed are variants of these and other proteins herein disclosed which have at least 50%, 51%, 52%, 53%, 54%, 55%, 56%, 57%, 58%, 59%, 60%, 61%, 62%, 63%, 64%, 65%, 66%, 67%, 68%, 69%, 70%, 71% 72%, 73%, 74%, 75%, 76%, 77%, 78%, 79%, 80% 81%, 82%, 83%, 84%, 85%, 86%, 87%, 88%, 89%, 90%, 91%, 92%, 93%, 94%, 95%, 96%, 97%, 98%, or 99% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman (Adv. Appl. Math. 2: 482 (1981), by the homology alignment algorithm of Needleman and Wunsch (J. Mol Biol. 48: 443 (1970)), by the search for similarity method of Pearson and Lipman (Proc. Natl. Acad. Sci. U.S.A. 85: 2444 (1988)), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52 (1989)_{;} Jaeger et al. Proc. Natl. Acad. Sci. USA 86:7706-7710 (1989), Jaeger et al. Methods Enzymol. 183:281-306 (1989) which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is understood that while no amino acid sequence indicates what particular DNA sequence encodes that protein within an organism, where particular variants of a disclosed protein are disclosed herein, the known nucleic acid sequence that encodes that protein in the particular sequence from which that protein arises is also known and herein disclosed and described.

It is understood that there are numerous amino acid and peptide analogs which can be incorporated into the disclosed compositions. For example, there are numerous D amino acids or amino acids which have a different functional substituent than the amino acids shown in Table 1 and Table 2. The opposite stereoisomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994)) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data, Vol. 1, Issue 3, Peptide Backbone Modifications (general review) (March 1983); Morley, Trends Pharm Sci pp. 463-468 (1980); Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al, Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

### C. Methods of Treatment and Prevention

Disclosed are methods of inhibiting the CXCR4 and/or the CCR5 receptors in a subject, comprising administering to the subject an effective amount of a GHS-R antagonist. Inhibiting can comprise blocking the binding of a ligand to CXCR4 and/or CCR5, for example.

1. Inflammation

Disclosed herein are methods of treating a subject with inflammation comprising administering to the subject an effective amount of a GHS-R antagonist. Inflammation is a complex stereotypical reaction of the body expressing the response to damage of cells and vascularized tissues. The discovery of the detailed processes of inflammation has revealed a close relationship between inflammation and the immune response. The main features of the inflammatory response are vasodilation, i.e. widening of the blood vessels to increase the blood flow to the infected area; increased vascular permeability, which allows diffusible components to enter the site; cellular infiltration by chemotaxis, or the directed movement of inflammatory cells through the walls of blood vessels into the site of injury; changes in biosynthetic, metabolic, and catabolic profiles of many organs; and activation of cells of the immune system as well as of complex enzymatic systems of blood plasma.

There are two forms of inflammation, acute and chronic. Acute inflammation can be divided into several phases. The earliest, gross event of an inflammatory response is temporary vasoconstriction, i.e. narrowing of blood vessels caused by contraction of smooth muscle in the vessel walls, which can be seen as blanching (whitening) of the skin. This is followed by several phases that occur minutes, hours and days later. The first is the acute vascular response, which follows within seconds of the tissue injury and lasts for several minutes. This results from vasodilation and increased capillary permeability due to alterations in the vascular endothelium, which leads to increased blood flow (hyperemia) that causes redness (erythema) and the entry of fluid into the tissues (edema).

The acute vascular response can be followed by an acute cellular response, which takes place over the next few hours. The hallmark of this phase is the appearance of granulocytes, particularly neutrophils, in the tissues. These cells first attach themselves to the endothelial cells within the blood vessels (margination) and then cross into the surrounding tissue (diapedesis). During this phase erythrocytes may also leak into the tissues and a hemorrhage can occur. If the vessel is damaged, fibrinogen and fibronectin are deposited at the site of injury, platelets aggregate and become activated, and the red cells stack together in what are called “rouleau” to help stop bleeding and aid clot formation. The dead and dying cells contribute to pus formation. If the damage is sufficiently severe, a chronic cellular response may follow over the next few days. A characteristic of this phase of inflammation is the appearance of a mononuclear cell infiltrate composed of macrophages and lymphocytes. The macrophages are involved in microbial killing, in clearing up cellular and tissue debris, and in remodeling of tissues.

Chronic inflammation is an inflammatory response of prolonged duration—weeks, months, or even indefinitely—whose extended time course is provoked by persistence of the causative stimulus to inflammation in the tissue. The inflammatory process inevitably causes tissue damage and is accompanied by simultaneous attempts at healing and repair. The exact nature, extent and time course of chronic inflammation is variable, and depends on a balance between the causative agent and the attempts of the body to remove it. Etiological agents producing chronic inflammation include: (i) infectious organisms that can avoid or resist host defenses and so persist in the tissue for a prolonged period, including Mycobacterium tuberculosis, Actinomycetes, and numerous fungi, protozoa and metazoal parasites. Such organisms are in general able to avoid phagocytosis or survive within phagocytic cells, and tend not to produce toxins causing acute tissue damage. (ii) Infectious organisms that are not innately resistant but persist in damaged regions where they are protected from host defenses. An example is bacteria which grow in the pus within an undrained abscess cavity, where they are protected both from host immunity and from blood-borne therapeutic agents, e.g. antibiotics. Some locations are particularly prone to chronic abscess formation, e.g. bone, and pleural cavities. (iii) Irritant non-living foreign material that cannot be removed by enzymatic breakdown or phagocytosis. Examples include a wide range of materials implanted into wounds (wood splinters, grit, metals and plastics), inhaled (silica dust and other particles or fibers), or deliberately introduced (surgical prostheses, sutures, etc.) Also included are transplants. Dead tissue components that cannot be broken down may have similar effects, e.g. keratin squames from a ruptured epidermoid cyst or fragments of dead bone (sequestrum) in osteomyelitis. (iv) In some cases the stimulus to chronic inflammation may be a normal tissue component. This occurs in inflammatory diseases where the disease process is initiated and maintained because of an abnormality in the regulation of the body's immune response to its own tissues—the so-called auto-immune diseases. This response is seen in elderly and aging subjects. (v) For many diseases characterized by a chronic inflammatory pathological process the underlying cause remains unknown. An example is Crohn's disease.

Inflammation and activation of innate immunity are common responses to replication incompetent adenoviruses (Ad) which are used as vectors for gene therapy (Jooss, K. Gene Ther. 10:955-963 (2003); Zaiss, A. K. J. Virol. 76:4580-4590, (2002)). The complement system is central to both innate immunity and inflammation (Walport, M. J. N Eng J Med 344:1058-1066 and1140-1144 (2001)). Because it is comprised of multiple membrane-bound and blood factors, the complement system is of particular relevance in delivery of vectors administered intravenously. In fact, Cichon et al. (Gene Ther 8:1794-1800 (2001)) showed complement was activated in a majority of human plasma samples when challenged with different adenoviral serotypes; complement activation was completely dependent on anti-Ad antibody (Cichon (2001)).

The complement mediated inactivation is a multistep enzymatic cascade which finally results in formation of a membrane attack complex (MAC) mediating the perforation of membranes and subsequent lysis of the invading organism. It is either initiated by antigen-antibody complexes (classical pathway) or via an antibody independent pathway which is activated by certain particular polysaccharides, viruses and bacteria (alternative pathway).

The early pro-inflammatory cascade can be initiated by endotoxin (also known as lipopolysaccharide, or LPS). LPS is one of the major constituents of the cell walls of gram-negative bacteria. Recognition of conserved microbial products, such as LPS, by the innate immune system leads to a variety of signal transduction pathways. These signal transduction pathways mediate the induction and secretion of cytokines that can regulate the level and duration of an inflammatory response. The systemic inflammatory response that accompanies endotoxic shock (caused by triggers such as the presence of LPS) is controlled by the levels of pro- and anti-inflammatory cytokines. Although the production of pro-inflammatory cytokines by cells of the innate immune system plays an important role in mediating the initial host defense against invading pathogens (O'Neill (2000), an inability to regulate the nature or duration of the host's inflammatory response can often mediate detrimental host effects as observed in chronic inflammatory diseases. Additionally, in the early stages of sepsis, the host's inflammatory response is believed to be in a hyperactive state with a predominant increase in the production of pro-inflammatory cytokines that mediate host tissue injury and lethal shock (Cohen (2002). In this regard, the ability to suppress pro-inflammatory cytokines and/or enhance anti-inflammatory cytokines, i.e. IL-10, has been shown to severely reduce the toxic effects of endotoxin (Berg (1995); Howard (1993).

Inflammatory cytokines released by immune cells have been shown to act on the central nervous system (CNS) to control food intake and energy homeostasis (Hart, B L. Neurosci. Biobehay. Rev. 12: 123-137 (1988)). Decrease in food intake or anorexia is one of the most common symptoms of illness, injury or inflammation (Kotler, D. P. Ann. Internal Med. 133: 622-634 (2000)). Cytokines such as IL-1 β, IL-6 and TNF-α have been implicated in wasting associated with inflammation (Ershler et al. Annu. Rev. Med. 51: 245-270 (2000)), chronic low-grade inflammation in aging (Bruunsgaard et al. Curr. Opin. Hematol. 8: 131-136 (2001), McCarty, M. F. Med. Hypotheses 52: 465-477 (1999)), and atherosclerosis (Bochkov et al. Nature. 419: 77-81 (2002)).

Inflammation can be associated with a number of different diseases and disorders. Examples of inflammation include, but are not limited to, inflammation associated with hepatitis, inflammation associated with the lungs, inflammation associated with burns, and inflammation associated with an infectious process. Inflammation can also be associated with liver toxicity, which can be associated in turn with cancer therapy, such as apoptosis induction or chemotherapy, or a combination of the two, for example.

The inflammation can be associated with an inflammatory disease. Examples of inflammatory disease include, but are not limited to, asthma, systemic lupus erythematosus, rheumatoid arthritis, reactive arthritis, spondyarthritis, systemic vasculitis, insulin dependent diabetes mellitus, multiple sclerosis, experimental allergic encephalomyelitis, Sjögren's syndrome, graft versus host disease, inflammatory bowel disease including Crohn's disease, ulcerative colitis, and scleroderma. Inflammatory diseases also includes autoimmune diseases such as myasthenia gravis, Guillain-Barré disease, primary biliary cirrhosis, hepatitis, hemolytic anemia, uveitis, Grave's disease, pernicious anemia, thrombocytopenia, Hashimoto's thyroiditis, oophoritis, orchitis, adrenal gland diseases, anti-phospholipid syndrome, Wegener's granulomatosis, Behcet's disease, polymyositis, dermatomyositis, multiple sclerosis, vitiligo, ankylosing spondylitis, Pemphigus vulgaris, psoriasis, dermatitis herpetiformis, Addison's disease, Goodpasture's syndrome, Basedow's disease, thrombopenia purpura, allergy, and cardiomyopathy.

The inflammation can also be associated with cancer. Examples of types of cancer include, but are not limited to, lymphoma (Hodgldns and non-Hodgkins) B-cell lymphoma, T-cell lymphoma, leukemia such as myeloid leukemia and other types of leukemia, mycosis fungoide, carcinoma, adenocarcinoma, sarcoma, glioma, astrocytoma, blastoma, neuroblastoma, plasmacytoma, histiocytoma, melanoma, adenoma, hypoxic tumour, myeloma, AIDS-related lymphoma or AIDS-related sarcoma, metastatic cancer, bladder cancer, brain cancer, nervous system cancer, squamous cell carcinoma of the head and neck, neuroblastoma, glioblastoma, ovarian cancer, skin cancer, liver cancer, squamous cell carcinomas of the mouth, throat, larynx, and lung, colon cancer, cervical cancer, breast cancer, cervical carcinoma, epithelial cancer, renal cancer, genitourinary cancer, pulmonary cancer, esophageal carcinoma, head and neck carcinoma, hematopoietic cancer, testicular cancer, colo-rectal cancer, prostatic cancer, and pancreatic cancer.

Activated cells can also be treated at the site of inflammation. “Activated cells” are defined as cells that participate in the inflammatory response. Examples of such cells include, but are not limited to, T-cells and B-cells , macrophages, NK cells, mast cells, eosinophils, neutrophils, Kupffer cells, antigen presenting cells, as well as vascular endothelial cells.

Inflammation can be caused by an infectious process in a subject. When the inflammation is associated with an infectious process, the infectious process can be associated with a viral infection. Examples of viral infections include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

When the inflammation is associated with an infectious process, the infectious process can be associated with a bacterial infection. The bacterial infection can be caused by either gram positive or gram negative bacterium. The gram positive bacterium can be selected from the group consisting of: M. tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthraces, B. subtilis, Nocardia asteroides, and other Nocardia species, Streptococcus viridans group, Peptococcus species, Peptostreptococcus species, Actinomyces israelii and other Actinomyces species, and Propionibacterium acnes.

The gram negative bacterium can be selected from the group consisting of: Clostridium tetani, Clostridium perfringens, Clostridium botulinum, other Clostridium species, Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteriacae, Brucella abortus and other Brucella species, Burkholderia cepacia, Burkholderia pseudomallei, Francisella tularensis, Bacteroides fragilis, Fusobascterium nucleatum, Provetella species and Cowdria ruminantium.

The above examples of grain positive and gram negative bacteria are not intended to be limiting, but are intended to be representative of a larger population including all gram positive and gram negative bacteria, as well as non-gram test responsive bacteria. Examples of other species of bacteria include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrlo, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Ernpedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella.

When the inflammation is associated with an infectious process, the infectious process can be associated with a parasitic infection. Examples of parasitic infections include, but are not limited to, Toxoplasma gondii, Plasmodium species such as Plasmodium falciparum, Plasmodium vivax, Plasmodium malariae, and other Plasmodium species, Trypanosoma brucei, Trypanosoma cruzi, Leishmania species such as Leishmania major, Schistosoma such as Schistosoma mansoni and other Shistosoma species, and Entamoeba histolytica.

When the inflammation is associated with an infectious process, the infectious process can be associated with a fungal infection. Examples of fungal infections include, but are not limited to, Candida albicans, Cryptococcus neoformans, Histoplama capsulatum, Aspergillus fumigatus, Coccidiodes immitis, Paracoccidiodes brasiliensis, Blastomyces dermitidis, Pneomocystis carnii, Penicillium marneffi, and Alternaria alternata.

Also disclosed are methods of inhibiting secretion of cytokines, comprising administering an effective amount of a GHS-R antagonist. For example, the cytokines can be inhibited at the site of inflammation. The cytokine can be expressed by cells selected from the group consisting of T-cells, B-cells, dendritic cells, and mononuclear cells.

Examples of cytokines and immunomodulatory agents that can be employed in the methods of this invention include, but are not limited to, those participating in humoral inflammation, such as IL-3, IL-4, IL-5, IL-6, IL-7, IL-9, IL-10, IL-13, and transforming growth factor-β (TGF-β), and those contributing to cellular inflammation such as IL-1, IL-2, IL-3, IL-4, IL-7, IL-9, IL-10, IL-12, interferons (IFNs), IFN-γ inducing factor (IGIF), TGF-β and TNF-α and -β. GHS-R antagonists can be used to modulate cytokines and/or immunomodulators according to the methods of this invention both to treat an acute episode of disease and/or to maintain the subject's condition in a non-inflammatory state.

Cytokines are proteins made by cells that affect the behavior of other cells. Cytokines made by lymphocytes are often called lymphokines or interleukins (IL). Cytokines act on specific cytokine receptors on the cells they affect. Binding of the receptor induces activity in the cell such as growth, differentiation, or death. Several cytokines play key roles in mediating acute inflammatory reactions, namely IL-1, TNF-a, IL-6, IL-11, IL-8 and other chemokines, GCSF, and GM-CSF. Of these, IL-1 (α and β) and TNF are extremely potent inflammatory molecules: they are the primary cytokines that mediate acute inflammation induced in animals by intradermal injection of bacterial lipopolysaccharide and two of the primary mediators of septic shock.

Chronic inflammation may develop following acute inflammation and may last for weeks or months, and in some instances for years. During this phase of inflammation, cytokine interactions result in monocyte chemotaxis to the site of inflammation where macrophage activating factors (MAF), such as IFN-γ, MCP-1, and other molecules then activate the macrophages while migration inhibition factors (MIF), such as GM-CSF and IFN-γ, retain them at the inflammatory site. The macrophages contribute to the inflammatory process by chronically elaborating low levels of IL-1 and TNF which are responsible for some of the resulting clinical symptoms such as anorexia, cachexia, fever, sleepiness, and leukocytosis. The cytokines known to mediate chronic inflammatory processes can be divided into those participating in humoral inflammation, such as IL-3, IL-4, IL-5, IL-6, IL-7, IL-9, IL-10, IL-13, and transforming growth factor-β (TGF-β), and those contributing to cellular inflammation such as IL-1, IL-2, IL-3, IL-4, IL-7, IL-9, IL-10, IL-12, interferons (IFNs), IFN-γ inducing factor (IGIF), TGF-β and TNF-α and -β (Feghali et al. Frontiers in Bioscience 2, d12-26 (Jan. 1, 1997)).

The production of pro-inflammatory cytokines by cells of the innate immune system plays an important role in mediating the initial host defense against invading pathogens. Furthermore, the inability to regulate the nature or duration of the host's inflammatory response can often mediate detrimental host effects as observed in chronic inflammatory diseases. For example, in the early stages of sepsis, the host's inflammatory response is believed to be in a hyperactive state with a predominant increase in the production of pro-inflammatory cytokines that mediate host tissue injury and lethal shock. Thus, the ability of the innate immune system to dictate the levels of pro- and anti-inflammatory cytokine production is critical in limiting or modulating the nature of the host inflammatory response.

2. Cancer/Atherosclerosis

Disclosed herein are methods of treating and preventing cancer in a subject, comprising administering to the subject an effective amount of a GHS-R antagonist. Also disclosed are methods of treating atherosclerosis. Solid tumour growth is generally angiogenesis (neovascularization)-dependent, and angiogenesis inhibitors have therefore been used as agents for the treatment of solid tumours and metastasis. Endothelial cells (EC) in the vasculature play an essential role in angiogenesis, and there is accordingly a need for therapeutic agents that target this activity. The proliferation, migration and differentiation of vascular endothelial cells during angiogenesis is understood to be modulated in both normal and disease states by the complex interactions of a variety of chemokines and chemokine receptors. CXCR4 is expressed on vascular EC, and in such cells is reportedly the most abundant receptor amongst all examined chemokine receptors (Gupta, et al, 1998).

When CXCR4 is genetically deleted from human breast cancer cells, the cancer cells do not grow at all in mice (Cancer Biology and Therapy, 3:12 1192-1199, 2004). The role of CXCR4 in cancer growth transcends their ability to spread to new tissues. CXCR4 expression is necessary to prevent cancer and leukemia cells from undergoing programmed cell death. If CXCR4 is not allowed to bind to SDP-1, the cells harboring CXCR4 undergo apoptosis.

SDF-1 is overexpressed in lymph nodes, bone, lung and liver, the very tissues that actively concentrate metastatic cancer cells of all kinds. SDF-1 can be soluble or attached to the membranes of certain cells. When SDF-1 binds CXCR4, it initiates a series of biochemical changes that keeps the CXCR4 cancer cells alive and further allows them to change shape so they can enter previously excluded tissues.

It has been demonstrated that CXCR4 was the only one of 14 chemokine receptors expressed on ovarian cancers. In addition, the presence of the receptor's ligand, CXCL12, was observed in ascitic fluid from ovarian cancer patients. Two ovarian cancer cell lines and primary ovarian cancer cells isolated from ascites fluid were used to show that CXCL12 added to cells in suboptimal growth conditions at concentrations of 50 to 100 ng/mL led to significant proliferation of the cells. Blocking the receptor by adding antibodies to it, however, prohibited the cell growth.

The chemokine also stimulated tritiated thymidine uptake, a sign that it stimulates DNA synthesis; increased the production of tumor necrosis factor-alpha; and prompted tumor cell invasion through extracellular matrix. The importance of CXCL12 in ovarian cancer was reinforced by the finding of intracellular CXCL12 protein in cells from 15 of 18 ovarian cancer biopsies. These included 10 of 11 serous tumors, two endometrioid ovarian cancers, and three of five clear cell cancers.

Upregulation CXCR4 is essential for human epithelial growth factor 2 (HER2)-mediated breast cancer metastasis (Hung et al., Cancer Cell 2004; 6:459-469). It has been established that CXCR4 plays an important role in HER2-mediated metastasis. It was found that CXCR4 expression was 2.8 times greater in HER2 transfectants of certain breast cancer cells than in vector control cells. Moreover, use of a monoclonal antibody that downregulated HER2 expression led to downregulation of CXCR4. Similar results were seen when RNA interference was used to deplete HER2 expression.

As disclosed above, metastasis appears to be mediated by stromal cell-derived factor-1a (SDF-1a), which is produced by target organs. SDF-1a sends homing signals to the CXCR4 receptors on the HER2 cancer cells.

Activation of the chemokine receptor CCR5 regulates p53 transcriptional activity in breast cancer cells through pertussis toxin-, JAK2-, and p38 mitogen-activated protein kinase-dependent mechanisms (Manes, S. et al., 2003). CCR5 blockade significantly enhanced proliferation of xenografts from tumor cells bearing wild-type p53, but did not affect proliferation of tumor xenografts bearing a p53 mutation. In parallel, data obtained in a primary breast cancer clinical series showed that disease-free survival was shorter in individuals bearing the CCR5Δ32 allele than in CCR5 wild-type patients, but only for those whose tumors expressed wild-type p53. These findings show that CCR5 activity influences human breast cancer progression.

3. HIV/AIDS

Disclosed are methods of treating a subject with a viral infection (e.g., HIV infection) or at risk for an infection comprising administering to the subject an effective amount of a GHS-R antagonist or a functional fragment thereof. As used throughout, administration of an agent described herein can be combined with various other therapies. For example, a subject with HIV may be treated concomitantly with protease inhibitors and other agents. Disclosed herein are methods of treating HIV in a subject, comprising administering to the subject an effective amount of a GHS-R antagonist or a fragment thereof. Also disclosed are methods of preventing HIV in a subject, comprising administering to the subject an effective amount of a GHS-R antagonist or a fragment thereof. Examples include D-Lys GHRP-6 (SEQ ID NO: 1).

The primary cellular receptor for HIV entry is CD4. However, expression of CD4 on a target cell is necessary but not sufficient for HIV entry and infection. Several chemokine receptors act as co-factors that allow HIV entry when co-expressed with CD4 on a cell surface. The first of these to be identified was CXCR4, or fusin, which is expressed on T cells (Feng et al., 1996). Co-expression of CXCR4 and CD4 on a cell allow T-tropic HIV isolates to fuse with and infect the cell. HIV gp120 interacts with both CD4 and CXCR4 to adhere to the cell and to effect conformational changes in the gp120/gp41 complex that allow membrane fusion by gp41. CXCR4 is expressed on many T cells, but usually not on macrophages and does not allow fusion by macrophage-tropic (M-tropic) HIV isolates (Feng et al., 1996). It is interesting to note that stimulation with some bacterial cell wall products upregulates CXCR4 expression on macrophages and allows infection by T-tropic strains of HIV (Moriuchi et al., 1998).

Shortly after the identification of CXCR4, another co-receptor was identified. CCR5, which is expressed on macrophages and on some populations of T cells, can also function in concert with CD4 to allow HIV membrane fusion (Deng et al., 1996; Dragic et al., 1996; Alkhatib et al., 1996). HIV gp120 binding to CCR5 is CD4-dependent, as antibody inhibition of CD4 can reduce binding to CCR5 by 87% (Trkola et al., 1996). M-tropic HIV isolates appear to use CCR5 as their co-receptor for infection both of macrophages and of some T cells. Individuals with certain mutations in CCR5 are resistant to HIV infection (Liu et al., 1996; Samson et al., 1996; Dean et al., 1996).

4. Treatment

The agents and methods disclosed herein are of benefit to subjects who are experiencing inflammation or are at risk for inflammation, subjects who are experiencing cancer, and subjects who are experiencing a viral infection, for example. Also disclosed are methods of treating any other disease or disorder in which CXCR4 and/or CCR5 play a role. Because the agents and methods disclosed herein reduce the severity or duration of inflammation, any subject that can benefit from a reduction in inflammation can be treated with the methods and agents disclosed herein.

The compositions comprising an agent disclosed herein in a pharmaceutically acceptable carrier may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals is to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein in its entirety for the methods taught.

The compositions may be in solution or in suspension (for example, incorporated into microparticles, liposomes, or cells). These compositions may be targeted to a particular cell type via antibodies, receptors, or receptor ligands. The following references are examples of the use of this technology to target specific proteins to given tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a. Pharmaceutically Acceptable Carriers

Administration of GHRP-6 or fragments thereof disclosed herein can occur in conjunction with other therapeutic agents. Thus, the agents of the present invention can be administered alone or in combination with one or more therapeutic agents. For example, a subject can be treated with the disclosed agent alone, or in combination with chemotherapeutic agents, antibodies, antivirals, steroidal and non-steroidal anti-inflammatories, conventional immunotherapeutic agents, cytokines, chemokines, and/or growth factors. Combinations may be administered either concomitantly (e.g., as an admixture), separately but simultaneously (e.g., via separate intravenous lines into the same subject), or sequentially (e.g., one of the compounds or agents is given first followed by the second). Thus, the term “combination” or “combined” is used to refer to either concomitant, simultaneous, or sequential administration of two or more agents.

Delivery of the agents disclosed herein can be used therapeutically in combination with a pharmaceutically acceptable carrier. Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including opthamalically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b. Dosages

The substances of the present invention can be delivered at effective amounts or concentrations. An effective concentration or amount of a substance is one that results in treatment or prevention of the disease or disorder. Beck et al (Life Sci. 2004 Dec. 10; 76(4):473-478), for example, can be used to determine effective dosages. Based on this study, dosages of 10-50 mg/kg body weight can be administered. One skilled in the art would know how to determine an effective concentration or amount according to methods known in the art, as well as provided herein. One of skill in the art can utilize in vitro assays to optimize the in vivo dosage of a particular substance, including concentration and time course of administration.

The dosage ranges for the administration of the substances are those large enough to produce the desired effect in which the symptoms of the disorder are affected. For example, the dosage range can be from 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20 21, 22, 23, 24, 25, 26, 27, 28, 29, 30 31, 32, 33, 34, 35, 36, 37, 38, 39, 40, 41, 42, 43, 44, 45, 46, 47, 48, 49, 50 51, 52, 53, 54, 55, 56, 57, 58, 59, or 60 mg/kg body weight of a GHS-R antagonist, for example, or any amount in between.

The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any contraindications. Dosage can vary, and can be administered in one or more dose administrations daily, for one or several days.

For example, to evaluate the efficacy of treatment of humans with a disorder characterized by inflammation with a substance that modulates cytokine activity, the following studies can be performed. Patients with active inflammation of, for example, the lung who have failed standard medical therapy, which can include prednisone and/or other immunomodulators known in the art (parenterally or orally) for control of the disorder can be selected. Drug efficacy can be monitored. Patients can be randomized to two different protocols. In one protocol, subjects can remain on initial medication and in the second protocol, subjects can have their medication tapered after receiving the substance that modulates cytokine activity, such as a GHS-R antagonist.

As described above, the agents disclosed herein can be administered together with other forms of therapy. For example, the molecules can be administered with antibodies, antibiotics, or other cancer treatment protocols as described above, or viral vectors. When the agent is in a vector, as described above, the vector containing the nucleic acid for therapeutic purposes can also contain a GHS-R antagonist or a fragment thereof.

c. Nucleic Acid Approaches for Delivery

The substances of the present invention, including SEQ ID NO: 1, can also be administered in vivo and/or ex vivo to patients or subjects as a nucleic acid preparation (e.g., DNA or RNA) that encodes a substance, such as SEQ ID NO: 1, such that the patient's or subject's own cells take up the nucleic acid and produce and secrete the encoded substances.

The nucleic acids of the present invention can be in the form of naked DNA or RNA, or the nucleic acids can be in a vector for delivering the nucleic acids to the cells, whereby the DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Prornega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, (1986)). The recombinant retrovirus can then be used to infect and thereby deliver to the infected cells nucleic acid encoding a broadly neutralizing antibody (or active fragment thereof) of the invention. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, (994)), adeno-associated viral (AAV) vectors (Goodman et al., Blood 84:1492-1500 (1994)), lentiviral vectors (Naidini et al., Science 272:263-267 (1996)), and pseudotyped retroviral vectors (Agrawal et al., Exper. Hematol. 24:738-747 (1996)). Physical transduction techniques can also be used, such as liposome delivery and receptor-mediated and other endocytosis mechanisms (see, for example, Schwartzenberger et al., Blood 87:472-478, (1996)) to name a few examples. This invention can be used in conjunction with any of these or other commonly used gene transfer methods.

As one example, if the antibody-encoding nucleic acid of the invention is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001 (1997); Alvarez and Curiel, Hum. Gene Ther. 8:597-613, (1997)). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector of the present invention, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. (1995)).

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

### Example 1

### The GHS-R Blocker D-[Lys3]GHRP-6 Serves as a Dual CXCR4/CCR5 Chemokine Receptor Antagonist

### Materials and Methods

Cell culture and Cell lines. Human CBMR5, HH cells and glioma cell lines U-118 and SW-1008 cells were obtained and cultured as described by the American Type Culture Collection (ATCC). Pheresis packs were prepared from 4 healthy male donors between 18 and 45 years age for the isolation of PBMCs and T cells. The average BMI of our donors was within the normal range (18.5-24.9). PBMCs were obtained by Ficoll-Hypaque density centrifugation. T cells were purified from PBMCs using human T cell enrichment columns (R&D Systems, Minneapolis, Minn., USA) via high-affinity negative selection according to the manufacturer's instructions. Flow cytometric analysis typically revealed greater than 90% purity.

Intracellular calcium mobilization. Measurement of intracellular calcium release in response to ghrelin and SDF-1 was performed as described previously (Dixit et al. J. Clin. Invest. 114, 57-66 (2004)). Cells were incubated in PBS containing 5 mM Fura-2 acetoxymethyl ester (Molecular Probes) for 30 minutes at room temperature. The cells were subsequently washed and then resuspended at 1×10⁶/ml in PBS. A total of 2 ml of the cell suspension was placed in a continuously stirring cuvette at room temperature in an LS50B spectrophotometer (Perkin-Elmer, Wellesley, Mass., USA). Labeled T cells were treated with SDF-1 (100 ng/ml) and along with DLG (Sigma-Aldrich) at various concentrations. Fluorescence was monitored at λₑₓ₁=340 nm, λₑₓ₂=380 nm, and λ_{cm}=510 nm. The data are presented as the relative ratio of fluorescence excited at 340 and 380 nm.

Fluorokine ligand binding. Fluorokine binding assay was performed as described previously (Nguyen and Taub Blood 99: 4298-4306 (2002)), briefly, biotinylated SDF-1α and MIP-1α (Fluorokine; R&D Systems) staining was performed according to R&D Systems' protocols, with slight modifications. The control or treated cells were resuspended in PBS at 4×10⁶/ml. Fifty microliters of cells was then mixed with 20 μl of 2.5 μg/ml biotinylated SDF-1α and MIP-1α or 5 μg/ml biotinylated soybean trypsin inhibitor and then incubated at 4° C. for 1 h. Fluorescein-conjugated avidin (10 μg/ml) was added (10-20 μl) to the cells and incubated for an additional 30 min at 4° C. After incubation, cells were washed with 1× RDF-1 buffer (R&D Systems) and then fixed with 2% paraformaldehyde in PBS before being analyzed on a FACScan (BD Biosciences).

Flow cytometry. CEM-R5 cells (1×10⁶) in PBS containing 2% FBS were added to 1-2 μg of mAbs and incubated for 30 min on ice. Cells were washed with PBS, resuspended in 100 μl of 20 μg/ml GAM-AF488, and incubated on ice for 30 min. Cells were then washed with PBS and fixed with 2% paraformaldehyde in PBS, followed by analysis on a FACScan. For the prefixing experiments, cells were washed with PBS after DLG treatment and then fixed with 2% paraformaldehyde in PBS for 30 min on ice. After incubation, the cells were then washed with PBS, resuspended in PBS containing 2% FBS, and then incubated an additional 30 min on ice before staining with mAbs.

Western blot analysis. Control and treated cells were lysed in RIPA buffer supplemented with protease and phosphatase inhibitor cocktail (Sigma-Aldrich, St. Louis, Mo.) and protein concentrations of cell lysates were determined by Bradford assay. Protein lysates (30 μg) were diluted with sample buffer and separated on 4-20% Tris HCl SDS-polyacrylamide gels (Biorad, Hercules, Calif.) and electrophoretically transferred to nitrocellulose membranes (Schleicher & Schuell). The blots were then incubated with rabbit anti-phospho-P42 and pERK (Cell Signaling, Beverly, Mass.). Immune complexes were visualized by incubation with either an anti-rabbit or an anti-mouse HRP-conjugated secondary antibody (Amersham, Piscataway, N.J.). The immunoreactive band was visualized by enhanced chemiluminescence (Perkin-Elmer).

Chemotaxis Assay. Fluorescence-based Transwell chemotaxis assays were performed to assess cell migration. Primary human T cells, CEMR5 and HH cells were labeled with 10 μg/ml Hoechst 33342 (Molecular Probes) in cRPMI for 30 min at 37° C. The cells were then resuspended in RPMI with 1% FBS to a concentration of 1×10⁷/ml and 100 ul of Hoechst labeled cells (10⁶ cells) were added into the top chamber of the transwells. Cells were treated with DLG in the upper chamber and RPMI (0.6 ml) containing 1% FBS was added to the bottom wells of the 24-well plate with 100 ng/ml of SDF-1α. Transwell chambers with 5 μm pore filters (Corning CoStar, Acton, Mass.) were then placed into the wells. Cells (1×10⁶ in 100 μl) were then added to the chambers. After 2 h, the migrated cells in the bottom wells were transferred to triplicate wells of a 96-well plate in 150 μl volumes. Hoechst fluorescence was measured on a Fluoroskan Ascent FL fluorescence plate reader (Thermo Labsystems, Franklin, Mass.) at λₑₓ=355 nm, and λₑₘ=460 nm. Results are expressed as migration index calculated by subtracting the fluorescence intensity of media alone and comparing the values to the fluorescence intensity (relative number) of cells migrated into the bottom chamber in media alone, which is normalized to a value of 1. Fluorescence values were within the linear range of a standard dilution curve.

### Results

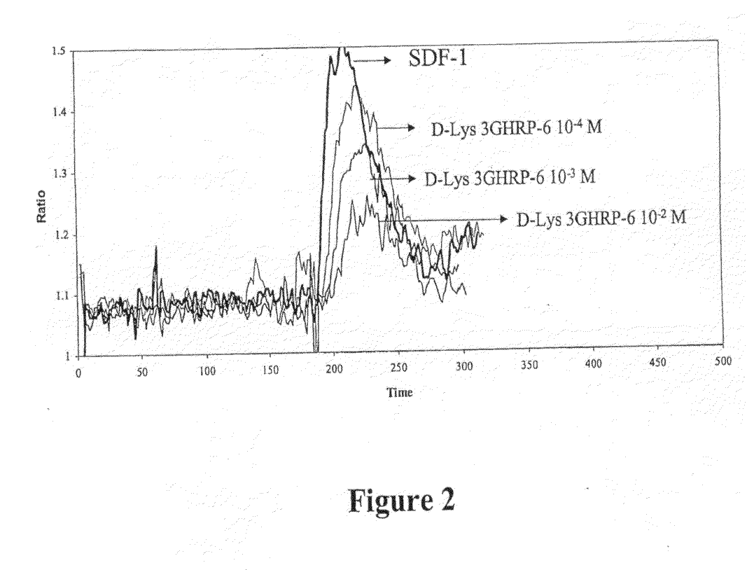

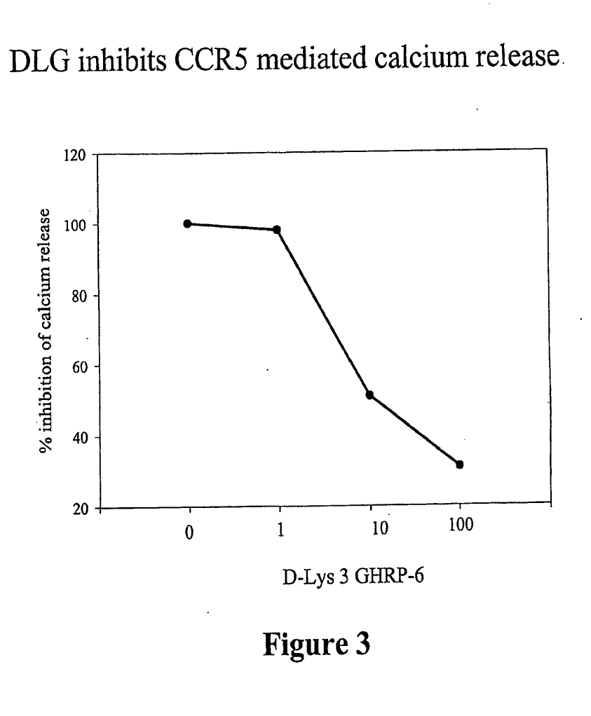

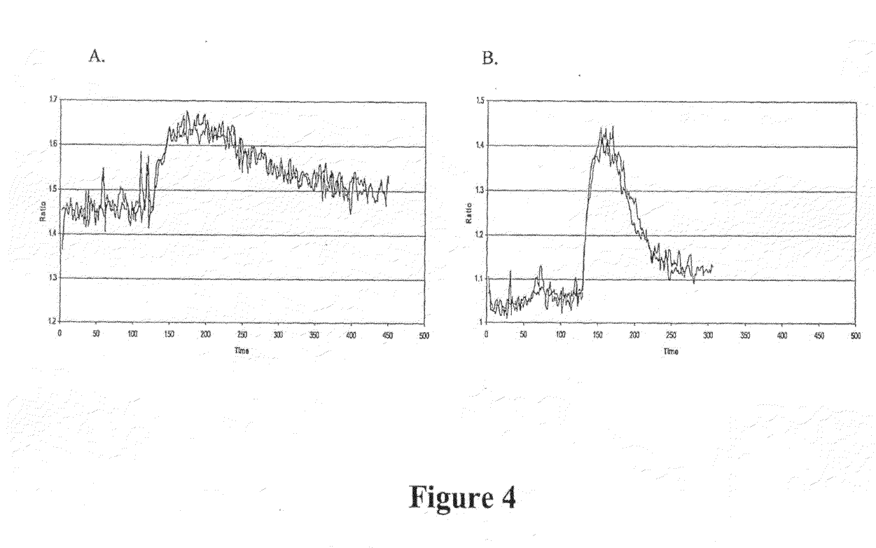

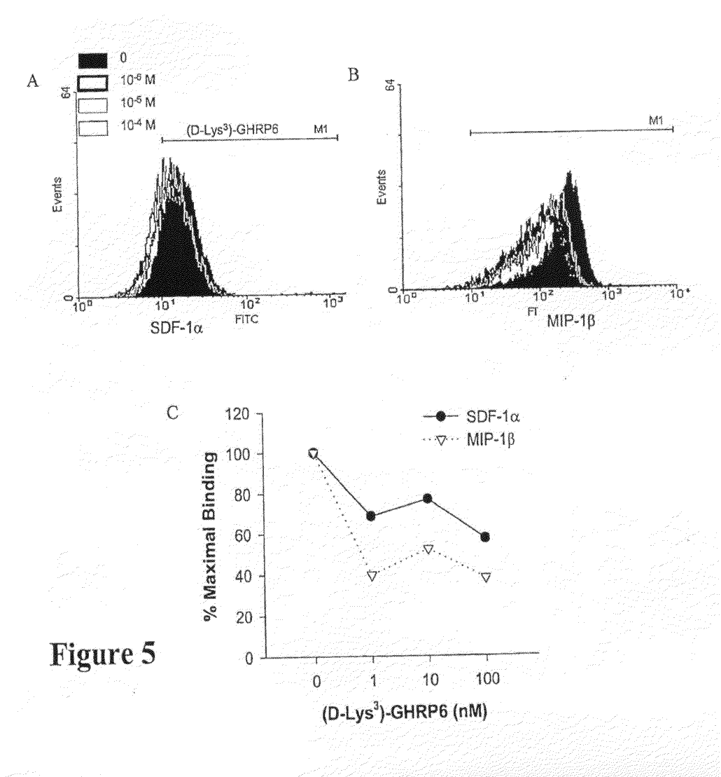

D-Lys3-GHRP-6 inhibits SDF-1 induced intracellular calcium release from human T Ligation of seven transmembrane GPCRs typically results in calcium mobilization from the intracellular stores by generation ofinositol triphosphate. SDF-1 binding to CXCR4 is known to elicit a potent release of calcium from intracellular sources. The direct effects of DLG on SDF-1 induced calcium mobilization were evaluated on primary human T cells labeled with Fura-2AM. SDF-1 at dose of 100 ng/ml caused a significant increase in intracellular calcium and this SDF-1-induced calcium flux was markedly inhibited in a dose dependent fashion by DLG (FIG. 2). Interestingly, the DLG also led to a dose dependent inhibition of calcium release post MLP-1β-CCR5 interaction in CEMR5 cells (FIG. 3). In addition, the DLG did not affect MIP-β (FIG. 4) and SIP (FIG. 5) mediated calcium flux in these cells.

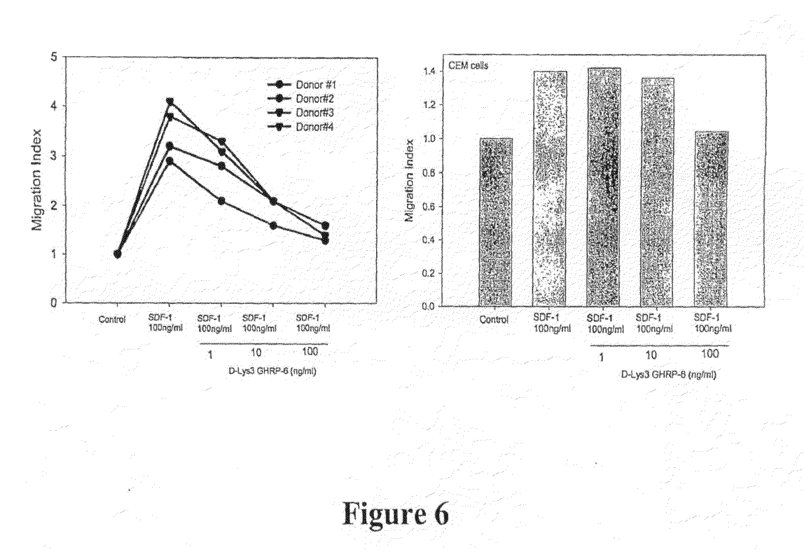

DLG inhibits SDF-1 and MIP-1β binding. To determine if DLG blocks CXCR4 and CCR5 receptors, a whole cell ligand binding assay was utilized with a biotinylated forms of SDF-1 and MIP-1β, followed by binding of an avidin-fluorescein isothiocyanate (FITC) conjugate, and subsequent examination by using flow cytometric analysis. Upon DLG treatment the percent maximal binding of SDF-1 and MIP-1β in the T-SUP1 cells was reduced by 50 to 60% (FIG. 6, 7) suggesting that DLG directly blocks the CXCR4 and CCR5 receptors.

DLG inhibits SDF-1 induced T cell chemotaxis. The functional consequences of inhibition of SDF-1 binding to CXCR4 by DLG were determined using a Transwell chamber migration assay. Primary human T cells upon optimal SDF-1 treatment (100 ng/ml) exhibited robust chemotaxis and treatment with DLG led to marked dose dependent inhibition of SDF-1 induced T cell migration. Similarly, DLG also inhibited the migration of CEMR5 human T cell lymphoma cell in response to CXCR4 migation by SDF-1.

DLG inhibits SDF-1 induced signaling in human astrocytoma cells. Many cancer cells utilize the CXCR4 chemokine receptors for their growth, development and impart migratory direction to these cells and thus play a critical role in orchestrating complex processes of tumor invasion and metastasis. Astrocytoma is the most common form of human brain tumor and astrocytoma and glioma cells express CXCR4 receptors for their growth, angiogenesis and invasion. It has been demonstrated that AMD3100 a clinically used CXCR4 antagonist can effectively inhibit intracranial growth of primary brain tumors via an ERK dependent pathways. It was observed that SDF-1 led to rapid phosphorylation and activation of ERK and P42 within 5 min that was sustained for 60 min, interestingly DLG abrogated the SDF-1 induced signaling in both SW1008 and U118 astrocytoma cells (FIG. 10).

### Discussion

Migration of immune cells to sites of inflammation is a multi-step process mediated largely by interactions of various chemokines to their G protein linked seven transmembrane receptors (Miyasaka et al. (2004), Campbell et al. (2003)). CXCR4 and CCR5 are the principal chemokine receptors critical for cellular migration and are used in association with CD4 by human immunodeficiency virus (HIV) to enter its target cells. These coreceptors are important determinants of viral tropism, pathogenesis and virulence and are widely believed to be important drug targets to prevent HIV infections (Castagna et al. (2005)). Currently AMD3100 a selective CXCR4 inhibitor has been successfully utilized to block CXCR4 mediated HIV viral entry (De Clercq E. Nat Rev Drug Discov. 2:581-587 (2003)), blocks glioma cell invasion (Rubin et al. (2003)), metastasis of breast (Smith et al. Cancer Res. 64: 8604-8612 (2004)) and pancreatic carcinoma (Marchesi et al. Cancer Res. 64: 8420-8427 (2004)), and decreases allergy and collagen induced arthritis (Lenoir Perianin A. J Immunol. 172: 7136-7143 (2004)). The synthetic peptidyl compound D-[Lys3]GHRP-6 (H-His-D-Trp-D-Lys-Trp-D-Phe-Lys-NH2) is believed to be a selective antagonist of ghrelin receptors (GHS-R). DLG can also antagonize the binding and signaling of CXCR4 and CCR5 chemokine receptors in human T cells as well as astrocytoma cancer cells. DLG has been utilized experimentally in rodent models without any adverse side effects, and repeated administration has been found to reduce body weight in obese mice and improve their glycaemic control and insulin resistance (Asakawa et al. Gut. 52, 947-952 (2003)). Furthermore, DLG reduced the size of abdominal fat pads without affecting the muscle mass in these mice. There is clinical evidence linking currently used HIV inhibitors to the pathogenesis of insulin resistance, dyslipidemia, lipodystrophy and atherosclerosis in AIDS patients (Kino and Chrousos Curr Drug Targets Immune Endocr Metabol Disord. 3: 111-117 (2003)). Thus, DLG along with its potential HW inhibitory properties may attenuate the metabolic effects associated with HAART therapy in AIDS patients. Additionally, DLG does not affect food intake in the fed state when circulating ghrelin levels are low, allowing for its potential use post-prandially.

### Example 2

### DLG Leads to Substantial Reduction in HIV Infectivity

Neomycin-resistant indicator CEM-GFP cells were used to monitor the infections with HIV1 (CXCR4, S1 strain). Viral entry into CEM cells via CXCR4 results in generation of green fluorescent protein signal. CEM cells (1 million/ml) were pretreated with DLG (1 ug/ml) and SDF-1 (1 ug/ml) for 30 min followed by 90 min viral incubation.

Generation of GFP fluorescence intensity was measured using fluorscan, compared to untreated cells, DLG led substantial reduction in HIV infectivity similar to the positive control SDF-1 (CXCL12).

Throughout this application, various publications are referenced. The disclosures of these publications in their entireties are hereby incorporated by reference into this application in order to more fully describe the state of the art to which this invention pertains.

### REFERENCES

Akoum, A., Lemay, A., McColl, S., Turcot-Lemay, L. and Maheux, R. (1996) Elevated concentration and biologic activity of monocyte chemotactic protein-1 in the fluid of patients with endometriosis. Fertil. Steril., 66, 17-23.

Alkhatib G, Combadiere C, Broder C C, Feng Y, Kennedy P E, Murphy P M, Berger E A. CC CKR5: a RANTES, MIP-1alpha, MIP-1beta receptor as a fusion cofactor for macrophage-tropic HIV-1. Science 1996 Jun. 28; 272(5270):1955-8.

Arici, A., Head, J. R., MacDonald, P. C. et al. (1993) Regulation of interleukin-8 gene expression in human endometrial cells in culture. Mol. Cell. Endocrinol., 94, 195-204.

Arici, A., McDonal, P. C. and Casey, M. L. (1995) Regulation of monocyte chemotactic protein-1 gene expression in human endometrial cells in cultures. Mol. Cell. Endocrinol., 107, 189-197.

Arici, A., Seli, E., Senturk, L. M., Gutierrez, L. S., Oral, E. and Taylor, H. S. (1998) Interleukin-8 in the human endometrium. Clin. Endocrinol. Metab., 83, 1783-1787.

Baggiolini, M. and Dahinden, A. (1994) CC chemokines in allergic inflammation. Immunol. Today, 15, 127-133.

Balkwill F. (2004). Cancer and the chemokine network. Nat Rev Cancer. 7: 540-550.

Barbero S, Bonavia R, Bajetto A, Porcile C, Pirani P, Ravetti I L, Zona G L, Spaziante R, Florio T, Schettini G. (2003). Stromal cell-derived factor 1alpha stimulates human glioblastoma cell growth through the activation of both extracellular signal-regulated kinases 1/2 and Akt. Cancer Res. 63:1969-1974.

Barclay, C. G., Brennard, J. E., Kelly, R. W. and Calder, A. A. (1993) Interleukin-8 production by human cervix. Am. J. Obstet. Gynecol., 169, 625-632.

Bloor, D. J., Metcalfe, A. D., Rutherford, A., Brison, D. R. and Kimber, S. J. (2002) Expression of cell adhesion molecules during human preimplantation embryo development. Mol. Hum. Reprod., 8, 237-245.

Bokoch, G. M. (1995) Chemoattractant signalling and leukocyte activation. Blood, 86, 1649-1657.

Broxmeyer H E, Orschell C M, Clapp D W, Hangoc G, Cooper S, Plett P A, Liles W C, Li X, Graham-Evans B, Campbell T B, Calandra G, Bridger G, Dale D C, Srour B F. (2005). Rapid mobilization of murine and human hematopoietic stem and progenitor cells with AMD3100, a CXCR4 antagonist. J Exp Med. 201:1307-1318.

Asakawa, A., Inui, A., Kaga, T., Katsuura, G., Fujimiya, M., Fujino, M. A., Kasuga, M. 2003. Antagonism of ghrelin receptor reduces food intake and body weight gain in mice. Gut. 52, 947-952.

Butcher, B. C. (1991) Leukocyte-endothelial cell recognition: three (or more) steps to specificity and diversity. Cell, 67, 1033-1036.

Caballero-Campo, P., Dominguez, F., Coloma, J., Meseguer, M., Remohi, J., Pellicer, A. and Simon, C. (2002) Hormonal and embryonic regulation of chemokines IL-8, MCP-1 and RANTES in human endometrium during the window of implantation. Mol. Hum. Reprod., 8, 375-384.

Campbell, J. J., Hedrick, J. Zlotnik, A., Siani, M. A., Thompson, D. A. and Butcher, E. C. (1998) Chemokines and the arrest of lymphocytes rolling under flow conditions. Science, 279, 381-384.

Campbell D J, Kim C H, Butcher E C. (2003). Chemokines in the systemic organization of immunity. Immunol Rev. 195: 58-71.

Cascieri M A, Springer M S. (2000) The chemokine/chemokine-receptor family: potential and progress for therapeutic intervention. Curr Opin Chem Biol. 4: 420-427.

Castagna A, Biswas P, Beretta A, Lazzarin A. (2005). The appealing story of HIV entry inhibitors: from discovery of biological mechanisms to drug development. Drugs. 65: 879-904.

Chantakru, S., Kuziel, W. A., Maeda, N. and Croy, B. A. (2001) A study on the density and distribution of uterine natural killer cells at mid pregnancy in mice genetically-ablated for CCR2B, CCR 5 and the CCR5 receptor ligand, MIP-1 alpha. J. Reprod. Immunol., 49, 33-47.

Chomczynski, P. and Sacchi, N. (1987) Single step method of RNA isolation by acid guanidinium thiocyanate phenol-chloroform extraction. Anal. Biochem., 162, 156-159.

Cocchi, F., DeVico, A. L., Garzino-Demo, A., Arya, S. K., Gallo, R. C. and Lusso, P. (1995) Identification of RANTES, MIP-1 alpha, and MIP-1 beta as the major HIV-suppressive factors produced by CD8+ T cells. Science, 270, 1811-1815.

Dean M, Carrington M, Winkler C, et al. Genetic restriction of HIV-1 infection and progression to AIDS by a deletion allele of the CCR5 structural gene. Science. 1996; 273:1856-1862.

De Clercq E. (2003). The bicyclam AMD3100 story. Nat Rev Drug Discov. 2: 581-587.

De los Santos, M. J., Mercader, A., Frances, A., Portoles, E., Remohi, J., Pellicer, A. and Simon, C. (1996) Role of endometrial factors in regulating secretion of components of the immunoreactive human embryonic interleukin-1 system during embryonic development. Biol. Reprod., 54, 563-574.

Del Pozo, M. A., Sanchez-Mateos, P., Nieto, M. and Sanchez-Madrid, F. (1995) Chemokines regulate cellular polarization and adhesion receptor redistribution during lymphocyte interaction with endothelium and extracellular matrix. Involvement of cAMP signaling pathway. J. Cell Biol., 131, 495-508

Del Pozo, M. A., Cabañas, C., Montoya, Ager, A., Sanchez-Mateos, P. and Sanchez-Madrid, F. (1997) ICAM's redistributed by chemokines to cellular uropods as a mechanism for recruitment of T-lymphocytes. J. Cell Biol., 137, 1-16.

Deng H, Liu R, Ellmeier W, Choe S, Unutmaz D, Burkhart M, Di Marzio P, Marmon S, Sutton R E, Hill C M, Davis C B, Peiper S C, Schall T J, Littman D R, Landau N R. Identification of a major co-receptor for primary isolates of HIV-1. Nature 1996 Jun. 20; 381(6584):661-6.

Dixit, V. D., Schaffer, E. M., Pyle, R. S., Collins, G. D., Sakthivel, S. K., Palaniappan, R., Lillard, I. W., Jr., Taub, D. D. 2004. Ghrelin inhibits leptin- and activation-induced proinflammatory cytokine expression by human monocytes and T cells. J. Clin. Invest. 114, 57-66.

Dominguez, F. et al. Hormonal and embryonic regulation of chemokine receptors CXCR1, CXCR4, CCR5 and CCR2B in the human endometrium and the human blastocyst. Molecular Human Reproduction, Vol. 9, No. 4, 189-198, April 2003.

Dragic T, Litwin V, Allaway G P, Martin S R, Huang Y, Nagashima K A, Cayanan C, Maddon P S, Koup R A, Moore J P, Paxton W A. HIV-1 entry into CD4+ cells is mediated by the chemokine receptor CC-CKR-5. Nature 1996 Jun. 20; 381(6584):667-73.

Dudley, D. J., Trantman, M. S. and Mitchel, M. D. (1993) Inflammatory mediators regulate interleukin-8 production by cultured gestational tissues: evidence for a cytokine network at the chorio-decidual interface. Clin. Endocrinol. Metab., 76, 404-410.

Feng Y, Broder C C, Kennedy P E, Berger E A. HIV-1 entry cofactor: functional cDNA cloning of a seven-transmembrane, G protein-coupled receptor. Science 1996 May 10; 272(5263):872-7.

Fernandis, J. et al. CXCR4/CCR5 Down-modulation and Chemotaxis Are Regulated by the Proteasome Pathway. J. Biol. Chem., Vol. 277, Issue 20, 18111-18117, May 17, 2002.

Galan, A., O'Connor, J. E., Valbuena, D., Herrer, R., Remohi, J., Pampfer, S., Pellicer, A. and Simon, C. (2000) The human blastocyst regulates endommtrial epithelial apoptosis in embryonic adhesion. Biol. Reprod., 63, 430-439.

Glasser, S. R., Mulholland, J. and Mani, S. K. (1991) Blastocyst endometrial relationships: reciprocal interaccions between uterine epithelial and stromal cells and blastocysts. Trophoblast Res., 5, 225-280.

He J, Chen Y, Farzan M, Choe H, Ohagen A, Gartner S, Busciglio I, Yang X, Hofmann W, Newman W, Mackay C R, Sodroski I, Gabuzda D. CCR3 and CCR5 are co-receptors for HIV-1 infection of microglia. Nature 1997 Feb. 13; 385(6617):645-9.

Hoogewerf, A. J., Kuschert, G. S. V., Proudfoot, A. E., Chung, C. W., Cooke, R. M., Hubbard, R. E., Wells, T. N. and Sanderson, P. N. (1997) Glycosaminoglycans mediate cell surface oligomerization of chemokines. Biochemistry, 36, 13570-13578.

Hornung, D., Ryan, I. P., Chao, V. A., Vigne, J. L., Schriock, E. D. and Taylor, R. N. (1997) Immunolocalization and regulation of the chemokine RANTES in human endometrial and endometriosis tissues and cells. Clin. Endocrinol., 82, 1621-1628.

Howard, A. D., Feighner, S. D., Cully, D. F., Arena, J. P., Liberator, P. A., Rosenblum, C. I., Hamelin, M., Hreniuk, D. L., Palyha, O. C., Anderson, J., Paress, P. S., Diaz, C., Chou, M., Liu, K. K., McKee, K. K., Pong, S. S., Chaung, L. Y., Elbrecht, A., Dashkevicz, M., Heavens, R., Rigby, M., Sirinathsinghji, D. J., Dean, D. C., Melillo, D. G., Patchett, A. A., Nargund, R., Griffin, P. R., DeMartino, J. A., Gupta, S. K., Schaeffer, J. M., Smith, R. G., Van der Ploeg, L. H. 1996. A receptor in pituitary and hypothalamus that functions in growth hormone release. Science. 273, 974-977.

Kao, L. C., Tulac, S., Lobo, S., Imani, B., Yang, J. P., Germeyer, A., Osteen, K., Taylor, R. N., Lessey, B. A. and Giudice, L. C. (2002) Global gene profiling in human endometrium during the window of implantation. Endocrinology, 143, 2119-2138.

Kayisli, U. A., Mahutte, N. G. and Arici, A. (2002) Uterine chemokines in reproductive physiology and pathology. Am. J. Reprod. Immunol., 47, 213-221.

Kino T, Cbrousos G P. (2003). AIDS-related insulin resistance and lipodystrophy syndrome. Curr Drug Targets Immune Endocr Metabol Disord. 3: 111-117.

Kojima, M., Kangawa, K. 2005. Ghrelin: structure and function. Physiol. Rev. 85, 495-522.

Larsen, C., Anderson, A., Aella, E., Oppenheim, J. J. and Matsushima, K. (1989) The neutrophil-activating protein (NAP-1) is also chemotactic for T lymphocytes. Science, 243, 1469-1466.

Lenoir M, Djerdjouri B, Perianin A. (2004). Stroma cell-derived factor 1alpha mediates desensitization of human neutrophil respiratory burst in synovial fluid from rheumatoid arthritic patients. J Immunol. 172: 7136-7143

Liu R, Paxton W A, Choe S, et al. Homozygous defect in HIV-1 coreceptor accounts for resistance of some multiply-exposed individuals to HIV-1 infection. Cell. 1996; 86:367-377.

Loetscher, P., Seitz, M., Clark-Lewis, I., Baggiolini, M. and Moser, B. (1994) Monocyte chemotactic proteins MCP-1, MCP-2, and MCP-3 are major attractants for human CD4+ and CD8+ T lymphocytes. FASEB J., 8, 1055.

Loetscher, P., Seitz, M., Clark-Lewis, I., Baggiolini, M. and Moser, B. (1996) Activation of NK cells by CC chemokines. Chemotaxis, Ca2+ mobilization, and enzyme release. S. Immunol., 156, 322.

Manes, S. et al., 2003. CCR5 Expression Influences the Progression of Human Breast Cancer in a p53-dependent Manner, Jnl. of Exp. Med., Vol. 198, Number 9, 1381-1389.

Marchesi F, Monti P, Leone B E, Zerbi A, Vecchi A, Piemonti L, Mantovani A, Allavena P. (2004) Increased survival, proliferation, and migration in metastatic human pancreatic tumor cells expressing functional CXCR4. Cancer Res. 64: 8420-8427.

Martin, J. C., Jasper, M. J., Valbuena, D., Meseguer, M., Remohi, J., Pellicer, A. and Simon, C. (2000) Increased adhesiveness in cultured endometrial-derived cells is related to the absence of moesin expression. Biol. Reprod., 63, 1370-1376.

Mellado, M., Rodriguez-Frade, J. M., Vila-Coro, A. J., Fernandez, S., Martin de Ana, A., Jones, D. R., Toran, J. L. and Martinez-A. C. (2001) Chemokine receptor homo- or heterodimerization activates distinct signaling pathways. EMBO J., 20, 2497-507.

Meseguer, M., Aplin, D., Caballero-Campo, P., O'Connor, J. E., Martin, J. C., Remohi, J., Pellicer, A. and Simon, C. (2001) Human endometrial mucin MUC1 is up-regulated by progesterone and down regulated in vitro by the human blastocyst. Biol. Reprod., 64, 590-601.

Miyasaka M, and Tanaka T. (2004). Lymphocyte trafficking across high endothelial venules: dogmas and enigmas. Nat Rev Immunol. 5: 360-370.

Moriuchi M, Moriuchi H, Turner W, Fauci A S. Exposure to bacterial products renders macrophages highly susceptible to T-tropic HIV-1. J Clin Invest 1998 Oct. 15; 102(8):1540-50.

Moser, B. and Loetscher, P. (2001) Lymphocyte traffic control by chemokines. Nat. Immunol., 2, 123-128.

Mukaida, N., Shiroo, M. and Matsushima, K. (1989) Genomic structure of the human monocyte derived neutrophil chemotactic factor IL-8. S. Immunol., 143, 1366-1371.

Nagasawa, T., Nakajima, T., Tachibana, K., Iizasa, H., Bleul, C. C., Yoshie, O., Matsushima, K., Yoshida, N., Springer, T. A. and Kishimoto, T. (1996) Molecular cloning and characterization of a murine pre-B-cell growth-stimulating factor/stromal cell-derived factor 1 receptor, a murine homolog of the human immunodeficiency virus 1 entry coreceptor fusin. Proc. Natl Acad. Sci. USA, 93, 14726-14729.

Nieto, M., Frade, J. M. R., Sancho, D., Mellado, M., Martinez-A, C. and Sanchez-Madrid, F. (1997) Polarization of chemokine receptors to the leading edge during lymphocyte chemotaxis. J. Exp. Med., 186, 153-158.

Nieto, M., Navarro, F., Perez-Villar, J. J., del Pozo, M. A., Gonzalez-Amaro, R., Mellado, M., Frade, J. M., Martinez-A, C., Lopez-Botet, M. and Sanchez-Madrid, F. (1998) Roles of chemokines and receptor polarization in NK-target cell interactions. S. Immunol., 161, 3330-3339.

Noyes, R. W., Hertig, A. T. and Rock, J. (1950) Dating the endometrial biopsy. Fertil. Steril., 1, 3-25.

Nguyen D and Taub D. (2002). Cholesterol is essential for macrophage inflammatory protein 1 beta binding and conformational integrity of CC chemokine receptor 5. Blood. 99: 4298-4306.

Payne, A. S. and Cornelius, L. A. (2002) The role of chemokines in melanoma tumor growth and metastasis. J. Invest. Dermatol., 118, 915-922.

Polentarutti, N., Allavena, P., Bianchi, G., Giardina, G., Basile, A., Sozzani, S., Mantovani, A. and Introna, M. (1997) IL-2-regulated expression of the monocyte chemotactic protein-1 receptor (CCR2B) in human NK cells: characterization of a predominant 3.4-kilobase transcript containing CCR2B and CCR2A sequences. J. Immunol., 158, 2689-2694.

Reif, K. and Cantrell, D. A. (1998) Networking Rho family GTPases in lymphocytes. Immunity, 8, 395-401.

Rodriguez-Frade, J. M., Mellado, M. and Martinez-A, C. (2001) Chemokine receptor dimerization: two are better than one. Trends Immunol., 22, 612-617.

Rubin J B, Kung A L, Klein R S, Chan J A, Sun Y, Schmidt K, Kieran M W, Luster A D, Segal R A. (2003) A small-molecule antagonist of CXCR4 inhibits intracranial growth of primary brain tumors. Proc Natl Acad Sci USA. 100: 13513-13518.

Saito, S., Kasahara, T., Sakakura, S., Umekage, H., Harada, N. and Ichijo, M. (1994) Detection and localisation of interleukin-8 mRNA and protein in human placenta and decidual tissues. J. Reprod. Immuol., 27, 161-172.

Samson M, Libert F, Doranz B J, et al. Resistance to HIV-1 infection of Caucasian individuals bearing mutant alleles of the CCR5 chemokine receptor gene. Nature. 1996; 382:722-725.

Sanchez-Madrid, F. and del Pozo, M. A. (1999) Leukocyte polarization in cell migration and immune interactions. EMBO J., 18, 501-511.

Sica, A., Warig, J. M., Colotta, F., Dejana, E., Mantovani, A., Oppenheim, J. J., Larsen, C. G., Zachariae, C. O. and Matsushima, K. (1990) Monocyte chemotactic and activating factor gene expression induced in endothelial cells by interleukin-1 (IL-1) and tumor necrosis factor (TNF-a). J. Immunol., 144, 3034-3038.

Simón, C., Piquette, G., Frances, A. and Polan, M. L. (1993) Localization of interleukin-1 type 1 receptor and interleukin-1b in human endometrium throughout the menstrual cycle. Clin. Endocrinol. Metab., 77, 549-555.

Simón, C., Piquette, G. N., Frances, A., el-Danasouri, I., Irwin, J. C. and Polan, M. L. (1994) The effect of interleukin-1b on the regulation of IL-1 receptor type 1 messenger ribonucleic acid and protein levels in cultured human endometrial stromal and glandular cells. Clin. Endocrinol. Metab., 78, 675-682.

Simón, C., Gimeno, M. J., Mercader, A., O'Connor, J. E., Remohi, J., Polan, M. L. and Pellicer, A. (1997) Embryonic regulation of integrins b3, a4 and a1 in human endometrial epithelial cells in vitro. Clin. Endocrinol. Metab., 82, 2607-2616.

Simón, C., Caballero-Campo, P., Garcia-Velasco, L. A. and Pellicer, A. (1998) Potential implications of chemokines in reproductive function: an attractive idea. J. Reprod. Immunol., 38, 169-193.

Simón, C., Mercader, A., Garcia-Velasco, J. A., Nikas, G., Moreno, C., Remohi, and Pellicer, A. (1999) Coculture of human embryos with autologous human endometrial epithelial cells in patients with implantation failure. Clin. Endocrinol. Metab., 84, 2638-2646.

Smith R G. (2005) Development of growth hormone secretagogues. Endocr Rev. 26: 346-360.

Smith M C, Luker K E, Garbow J R, Prior J L, Jackson E, Piwnica-Worms D, Luker G D. (2004). CXCR4 regulates growth of both primary and metastatic breast cancer. Cancer Res. 64: 8604-8612.

Trkola A, Dragic T, Arthos J, Binley J M, Olson W C, Allaway G P, Cheng-Mayer C, Robinson J, Maddon P J, Moore J P. CD4-dependent, antibody-sensitive interactions between HIV-1 and its co-receptor CCR-5. Nature 1996 Nov. 14; 384(6605):184-7.

Ward, S. G., Bacon, K. and Westwick, J. (1998) Chemokines and T lymphocytes: more than a attraction. Immunity, 9, 1-11.

Witt, D. P. and Lander, A. D. (1994) Differential binding of chemokines to glycosaminoglycan subpopulations. Curr. Biol., 4, 394.

Wuyts, A., Proost, P., Lenaerts, J. P., Ben-Baruch, A., Van Damme, J. and Wang, J. M. (1998) Differential usage of the CXC chemokine receptors 1 and 2 by interleukin-8, granulocyte chemotactic protein-2 and epithelial-cell-derived neutrophil attractant-78. Eur. J. Biochem., 255, 67-73.

Wyatt R, Sodroski J. The HIV-1 Envelope Glycoproteins: Fusogens, Antigens, and Immunogens. Science 1998; 280(5371): 1884-1888.

Yoshimura, T. and Leonard, E. J. (1990) Secretion by human fibroblasts of monocyte chemoattractant protein-1, the product of the gene JE. J. Immunol., 144, 2377-2383.

Yoshimura, T., Yuhki, N. and Moore, S. (1989) Human monocyte chemoattractant protein 1 (MCP-1): full-length cDNA cloning, expression in mitogen-stimulated blood mononuclear leukocytes and sequence similarity to mouse competence gene JE. FEBS Lett., 244, 487-493.

SEQUENCES
D-[Lys3]GHRP-6 (H-His-D-Trp-D-Lys-Trp-D-Phe-
Lys-NH2,)
D-[Arg3]GHRP-6 (H-His-D-Trp-D-Arg-Trp-D-Phe-
Lys-NH2)
D-[His3]GHRP-6 (H-His-D-Trp-D-His-Trp-D-Phe-
Lys-NH2)
D-[Ala3]GHRP-6 (H-His-D-Trp-D-Ala-Trp-D-Phe-
Lys-NH2

1. A method of blocking binding to a CXCR4 receptor in a subject comprising administering to the subject an effective amount of a GHS-R antagonist.

2. A method of blocking binding to a CCR5 receptor in a subject comprising administering to the subject an effective amount of a GHS-R antagonist.

3. A method of blocking binding to CCR5 and CXCR4 receptors in a subject comprising administering to the subject an effective amount of a GHS-R antagonist.

4. A method of blocking binding to CCR5 and CXCR4 receptors in a subject comprising administering to the subject an effective amount of SEQ ID NO: 1 or a fragment thereof.

5. The method of claim 1, wherein the GHS-R antagonist is selected from the group consisting of SEQ ID NO: 1, SEQ ID NO: 2, SEQ ID NO: 3, or SEQ ID NO: 4.

6. A method of treating a viral infection in a subject comprising administering to the subject an effective amount of SEQ ID NO: 1 or a fragment thereof.

7. A method of preventing a viral infection in a subject comprising administering to the subject an effective amount of SEQ ID NO: 1 or a fragment thereof.

8. A method of treating inflammation in a subject comprising administering to the subject an effective amount of SEQ ID NO: 1 or a fragment thereof.

9. The method of claim 8, wherein the inflammation is associated with an infectious process.

10. The method of claim 9, wherein the infectious process is a viral infection selected from the group consisting of Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency cirus, Human T-cell Leukemia virus type-1, Hantavirus, Rubella virus, Simian Immunodeficiency virus, Human Immunodeficiency virus type-1, and Human Immunodeficiency virus type-2.

11. The method of claim 9, wherein the infectious process is a bacterial infection selected from the group consisting of M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

12. The method of claim 9, wherein the infectious process is a parasitic infection selected from the group consisting of Toxoplasma gondii, Plasmodium, Trypanosoma brucei, Trypanosoma cruzi, Leishmania, Schistosoma, and Entamoeba histolytica.

13. The method of claim 9, wherein the infectious process is a fungal infection selected from the group consisting of Candida albicans, Cryptococcus neoformans, Histoplasma capsulatum, Aspergillus fumigatus, Coccidiodes immitis, Paracoccidiodes brasiliensis, Blastomyces dermitidis, Pneomocystis carnii, Penicillium marneffi, and Alternaria alternata.

14. The method of claim 8, wherein the inflammation is associated with an inflammatory disease.

15. The method of claim 14, wherein the inflammatory disease is selected from the group consisting of asthma, reactive arthritis, hepatitis, spondyarthritis, Sjögren's syndrome, Alzheimer's disease, sepsis, and atopic dermatitis.

16. The method of claim 14, wherein the inflammatory disease is associated with an autoimmune disease.

17. The method of claim 16, wherein the autoimmune disease is systemic lupus erythematosus, rheumatoid arthritis, systemic vasculitis, insulin dependent diabetes mellitus, multiple sclerosis, experimental allergic encephalomyelitis, psoriasis, Crohn's disease, inflammatory bowel disease, ulcerative colitis, Addison's disease, alopecia aretea, celiac disease, thyroid disease, and scleroderma.

18. The method of claim 8, wherein the inflammation is associated with a burn.

19. The method of claim 8, wherein the inflammation is associated with lung inflammation.

20. A method of treating cancer in a subject comprising administering to the subject an effective amount of SEQ ID NO: 1 or a fragment thereof.

21. The method of claim 20, wherein the cancer can be selected from the group consisting of lymphoma, leukemia, mycosis fungoide, carcinoma, adenocarcinoma, sarcoma, glioma, astrocytoma, blastoma, neuroblastoma, plasmacytoma, histiocytoma, melanoma, adenoma, hypoxic tumour, myeloma, AIDS-related lymphoma or AIDS-related sarcoma, metastatic cancer, bladder cancer, brain cancer, nervous system cancer, glioblastoma, ovarian cancer, skin cancer, liver cancer, squamous cell carcinomas of the mouth, throat, larynx, and lung, colon cancer, cervical cancer, breast cancer, epithelial cancer, renal cancer, genitourinary cancer, pulmonary cancer, esophageal carcinoma, head and neck carcinoma, hematopoietic cancer, testicular cancer, colo-rectal cancer, prostatic cancer, and pancreatic cancer.

22. A method of treating atherosclerosis in a subject comprising administering to the subject an effective amount of SEQ ID NO: 1 or a fragment thereof)

23. The method of claim 2, wherein the GHS-R antagonist is selected from the group consisting of SEQ ID NO: 1, SEQ ID NO: 2, SEQ ID NO: 3, or SEQ ID NO: 4.
